(19) United States
(12) Patent Application Publication
(10) Pub. No.: US 2022/0051271 A1
(43) Pub. Date: Feb. 17, 2022

(54) RECEIPT PROCESSING APPARATUS, CONTROL METHOD, AND PROGRAM

(71) Applicant: NEC Corporation, Minato-ku, Tokyo (JP)

(72) Inventor: Kouji KOBAYASHI, Tokyo (JP)

(73) Assignee: NEC Corporation, Minato-ku, Tokyo (JP)

(21) Appl. No.: 17/413,162

(22) PCT Filed: Nov. 20, 2019
(86) PCT No.: PCT/JP2019/045473

§ 371 Date: Jun. 11, 2021

(30) Foreign Application Priority Data
Dec. 20, 2018 (JP) 2018-238152

(51) Int. Cl. G06Q 30/02

(52) U.S. Cl. CPC G06Q 30/0202

(57) ABSTRACT

A receipt processing apparatus (2000) acquires receipt information (10) and target product information (20). The receipt information (10) indicates information relating to a purchased product. The receipt processing apparatus (2000) generates quantity information (30) indicating a purchased quantity or a purchased frequency of the target product indicated in the target product information (20) out of the products indicated in the receipt information (10). The receipt processing apparatus (2000) transmits the quantity information (30) to a predetermined transmission destination.

### TECHNICAL FIELD

The present invention relates to a technique for handling information relating to a purchased product.

### BACKGROUND ART

A technique for issuing receipt information representing purchase details at the time of purchasing a product has been developed. For example, PTL 1 discloses a technique for transferring receipt information to a customer who made shopping.

### RELATED DOCUMENT

### Patent Document

[Patent Document 1] Japanese Patent Application Publication No. 2003-077064

### SUMMARY OF THE INVENTION

### Technical Problem

The inventor of the present application found a novel utilization method of receipt information. One of objects of the present invention is to provide a novel technique for utilizing receipt information.

### Solution to Problem

A receipt processing apparatus according to the present invention includes: 1) a generation unit acquiring target product information indicating a target product, and receipt information relating to a purchased product, and generating, from a product indicated in the receipt information, quantity information indicating a purchased quantity or a purchased frequency of a target product indicated in the target product information; and 2) a transmission unit transmitting the quantity information to a predetermined transmission destination.

A control method according to the present invention is executed by a computer. The control method includes: 1) a generation step of acquiring target product information indicating a target product, and receipt information relating to a purchased product, and generating, from a product indicated in the receipt information, quantity information indicating a purchased quantity or a purchased frequency of a target product indicated in the target product information; and 2) a transmission step of transmitting the quantity information to a predetermined transmission destination.

A program according to the present invention causes a computer to execute each step included in the control method according to the present invention.

### Advantageous Effects of Invention

The present invention provides a novel technique for utilizing receipt information.

### BRIEF DESCRIPTION OF THE DRAWINGS

FIG. 1 is a diagram illustrating an overview of a receipt processing apparatus according to a present example embodiment.

FIG. 2 is a diagram illustrating a functional configuration of a receipt processing apparatus according to an example embodiment 1.

FIG. 3 is a diagram illustrating a computer for achieving the receipt processing apparatus.

FIG. 4 is a flowchart illustrating a flow of processing to be executed by the receipt processing apparatus according to the example embodiment 1.

FIG. 5 is a first diagram illustrating a usage pattern of the receipt processing apparatus.

FIG. 6 is a second diagram illustrating a usage pattern of the receipt processing apparatus.

FIG. 7 is a flowchart illustrating a flow of processing to be executed by a receipt processing apparatus according to an example embodiment 2.

### DESCRIPTION OF EMBODIMENTS

In the following, example embodiments according to the present invention are described with reference to the drawings. Note that, in all of the drawings, similar constituent elements are designated by similar reference signs, and description thereof is omitted as necessary. Further, in each block diagram, each block represents a configuration of a functional unit instead of a configuration of a hardware unit unless otherwise specifically described.

### Example Embodiment 1

### <Overview>

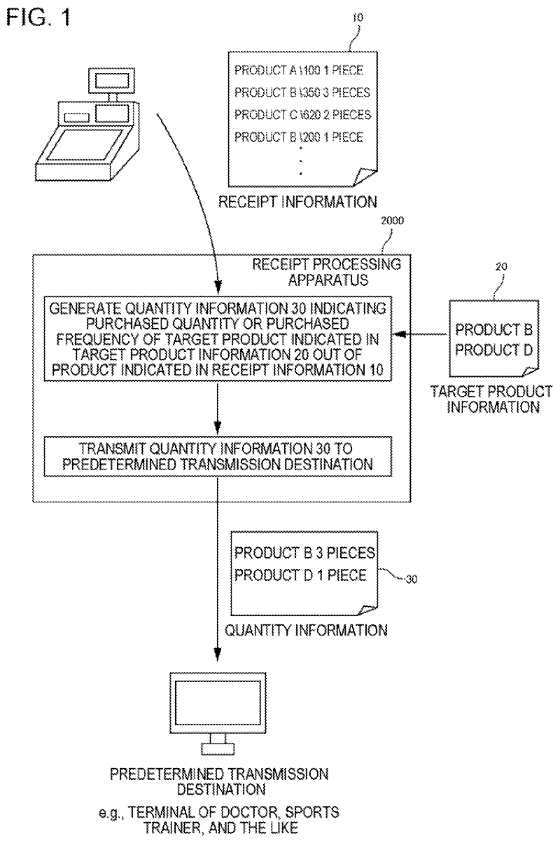

FIG. 1 is a diagram illustrating an overview of a receipt processing apparatus according to a present example embodiment. Note that, FIG. 1 is an illustration for making it easy to understand a receipt processing apparatus 2000, and a function of the receipt processing apparatus 2000 is not limited to the one illustrated in FIG. 1.

The receipt processing apparatus 2000 handles receipt information 10. The receipt information 10 is data indicating information relating to a purchased product. For example, in the example of FIG. 1, the receipt information 10 indicates a name, a price, and the number of pieces purchased, for each product. The receipt processing apparatus 2000 is, for example, a terminal such as a mobile terminal and a personal computer (PC) used by a person (hereinafter, a user) who purchased a product indicated in the receipt information 10. Hereinafter, a mobile terminal, a PC, and the like used by a user are generically referred to as a user terminal.

The receipt processing apparatus 2000 generates quantity information 30 indicating a quantity relating to a target product out of the products indicated the receipt information 10. Herein, the quantity relating to a product is, for example, a purchased quantity, a purchased frequency, and the like of the product. The target product is indicated in target product information 20. For example, in the example of FIG. 1, the target product information 20 indicates that a product B and a product D are the target products.

The receipt processing apparatus 2000 transmits the generated quantity information 30 to a predetermined transmission destination. Any destination may be adopted as the predetermined transmission destination. For example, the predetermined transmission destination is a terminal of a facility associated with health management and promotion of a user of the receipt processing apparatus 2000, or a terminal used by a person who performs health management and promotion of a user. The facility associated with health management and promotion of a user is, for example, a hospital, a sports gym, and the like where the user goes. Further, the person who performs health management and promotion of a user is, for example, a doctor, a sports trainer, and the like. In addition to the above, for example, the person who performs health management and promotion of a user may be a family member and the like of the user. For example, a case is proposed in which a family member living away from an elderly family member may wish to manage details of a meal and the like of the elderly family member.

Any product can be adopted as the target product. For example, the target product is a product associated with health management and promotion of a user, such as groceries (including beverages) and tobacco.

Note that, as described later, the receipt processing apparatus 2000 may transmit the quantity information 30 each time one piece of the receipt information 10 is acquired, or transmit the quantity information 30 (e.g., quantity information 30 indicating an integrated value of purchased quantities), which is obtained by collecting information acquired from a plurality of pieces of the receipt information 10.

### Advantageous Effect

As described above, in the receipt processing apparatus 2000 according to the present example embodiment, the quantity information 30 relating to a target product included in the receipt information 10 is transmitted to a predetermined transmission destination. In this way, the receipt processing apparatus 2000 provides a novel utilization method of receipt information that “the receipt information 10 is utilized to make it easy to manage a purchased quantity and the like of a target product purchased by a user”.

For example, a purchased quantity and the like of a target product associated with health management and promotion of a user are caused to transmit to a terminal in a facility and the like used for health management and promotion of a user. Thus, health management/promotion of a user can be more easily and accurately performed. Further, transmitting, by the receipt processing apparatus 2000, a purchased quantity and the like of a target product as described above allows a user to voluntarily refrain from purchasing the target product, consequently, an advantageous effect that user's health is promoted can also be expected.

Further, in the receipt processing apparatus 2000 according to the present example embodiment, the quantity information 30 indicates a purchased quantity and the like of only a target product. Specifically, a purchased quantity and the like of a product other than the target product are not included in the quantity information 30. Therefore, specifying, as a target product, only a product associated with a purpose of use of the quantity information 30 enables to exclude, from the quantity information 30, a purchased quantity of a product that is not associated with the purpose of use. Therefore, it is possible to achieve health management and the like of a user, while considering privacy of the user.

In the following, the present example embodiment is described in further detail.

### Example of Functional Configuration

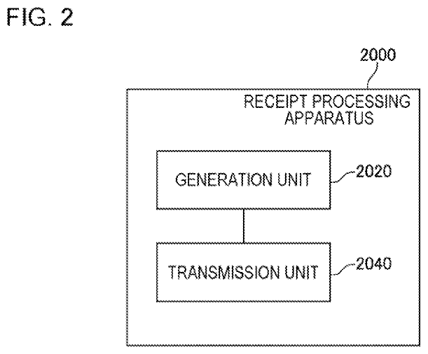

FIG. 2 is a diagram illustrating a functional configuration of the receipt processing apparatus 2000 according to an example embodiment 1. The receipt processing apparatus 2000 includes a generation unit 2020 and a transmission unit 2040. The generation unit 2020 acquires receipt information 10 and target product information 20. Further, the generation unit 2020 generates quantity information 30 for the target product indicated in the target product information 20 out of the products indicated in the receipt information 10. The transmission unit 2040 transmits the generated quantity information 30 to a predetermined transmission destination.

### Example of Hardware Configuration of Receipt Processing Apparatus 2000

Each functional configuration unit of the receipt processing apparatus 2000 may be achieved by hardware (example: a hard-wired electronic circuit, and the like) for achieving each functional configuration unit, or may be achieved by combination of hardware and software (example: combination of an electronic circuit and a program for controlling the electronic circuit, and the like). In the following, a case where each functional configuration unit of the receipt processing apparatus 2000 is achieved by combination of hardware and software is further described.

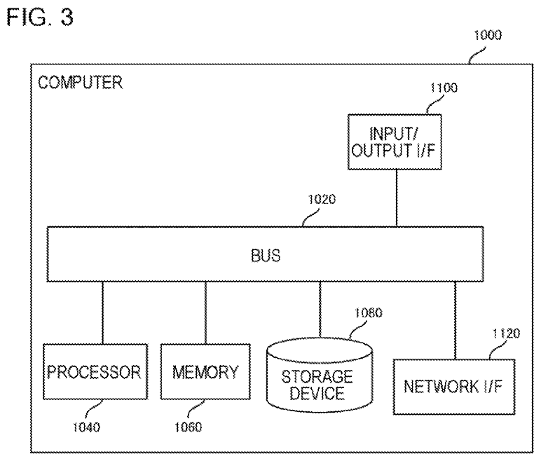

FIG. 3 is a diagram illustrating a computer 1000 for achieving the receipt processing apparatus 2000. The computer 1000 is any computer. For example, the computer 1000 is a stationary computer such as a personal computer (PC) and a server machine. In addition to the above, for example, the computer 1000 is a portable computer such as a smartphone and a tablet terminal. Note that, the computer 1000 may be a dedicated computer designed for achieving the receipt processing apparatus 2000, or may be a general-purpose computer.

The computer 1000 includes a bus 1020, a processor 1040, a memory 1060, a storage device 1080, an input/output interface 1100, and a network interface 1120. The bus 1020 is a data transmission path along which the processor 1040, the memory 1060, the storage device 1080, the input/output interface 1100, and the network interface 1120 mutually transmit and receive data. However, a method of mutually connecting the processor 1040 and the like is not limited to bus connection.

The processor 1040 is a variety of processors such as a central processing unit (CPU), a graphics processing unit (GPU), and a field-programmable gate array (FPGA). The memory 1060 is a main storage apparatus to be achieved by using a random access memory (RAM) or the like. The storage device 1080 is an auxiliary storage apparatus to be achieved by using a hard disk, a solid state drive (SSD), a memory card, a read only memory (ROM), or the like.

The input/output interface 1100 is an interface for connecting the computer 1000 to an input/output device. For example, the input/output interface 1100 is connected to an input apparatus such as a keyboard, and an output apparatus such as a display apparatus.

The network interface 1120 is an interface for connecting the computer 1000 to a communication network. The communication network is, for example, a local area network (LAN) or a wide area network (WAN). A method of connecting to a communication network by the network interface 1120 may be wireless connection or may be wired connection.

The storage device 1080 stores a program module for achieving each functional configuration unit of the receipt processing apparatus 2000. The processor 1040 achieves a function associated with each program module by reading each program module in the memory 1060 and executing the program module.

### <Flow of Processing>

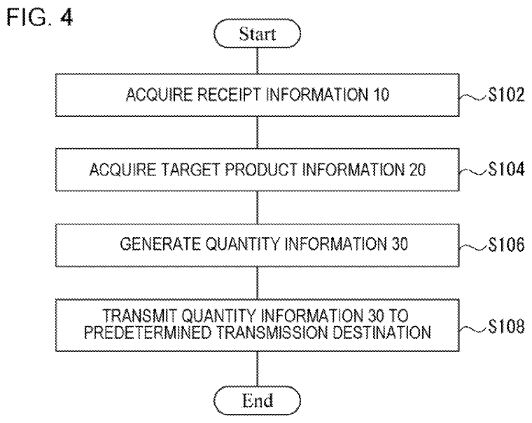

FIG. 4 is a flowchart illustrating a flow of processing to be executed by the receipt processing apparatus 2000 according to the example embodiment 1. The generation unit 2020 acquires receipt information 10 (S102). The generation unit 2020 acquires target product information 20 (S104). The generation unit 2020 generates quantity information 30 about a target product included in the receipt information 10 (S106). The transmission unit 2040 transmits the generated quantity information 30 to a predetermined transmission destination (S108).

Herein, there are a variety of triggers by which the receipt processing apparatus 2000 performs a series of processing indicated in FIG. 4. For example, the receipt processing apparatus 2000 receives the receipt information 10 in response to transmission of the receipt information 10 from another apparatus (S102), and performs each processing thereafter accompanied by the reception. In addition to the above, for example, the receipt processing apparatus 2000 may perform the series of processing illustrated in FIG. 4 in response to accept of a predetermined user operation.

### Example of Usage Pattern of Receipt Processing Apparatus 2000

For example, as described above, the receipt processing apparatus 2000 is achieved by a terminal used by a user who purchased a product indicated in the receipt information 10. However, the implementation pattern of the receipt processing apparatus 2000 is not limited to this example. Herein, several implementation patterns of the receipt processing apparatus 2000 and usage patterns thereof are described for making it easy to understand the receipt processing apparatus 2000.

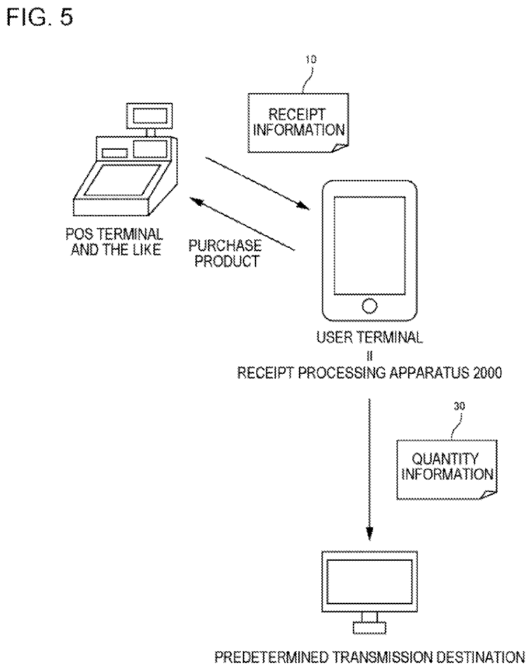

FIG. 5 is a first diagram illustrating a usage pattern of the receipt processing apparatus 2000. In this example, the receipt processing apparatus 2000 is achieved by a user terminal. For example, a user installs, in a user terminal, an application for achieving a function of the receipt processing apparatus 2000. Thus, the user terminal is functioned as the receipt processing apparatus 2000.

The receipt information 10 is transmitted to the receipt processing apparatus 2000 at the time of purchasing a product by a user. The receipt processing apparatus 2000 generates the quantity information 30 by processing the acquired receipt information 10, and transmits the generated quantity information 30 to a predetermined transmission destination.

For example, a user purchases a product by using the receipt processing apparatus 2000. Specifically, the user makes payment by performing near field communication between the receipt processing apparatus 2000 and a point of sales (POS) terminal at a shop, or does online shopping by using the receipt processing apparatus 2000. Consequently, the receipt processing apparatus 2000 acquires the receipt information 10 from the POS terminal, a Web server, or the like.

However, a user does not have to purchase a product by using the receipt processing apparatus 2000, and may make payment by cash. In this case, the receipt processing apparatus 2000 is registered in advance as a transmission destination of the receipt information 10. For example, a user may register a transmission destination of the receipt information 10 at the time of making membership registration or account registration at a shop or a shopping site.

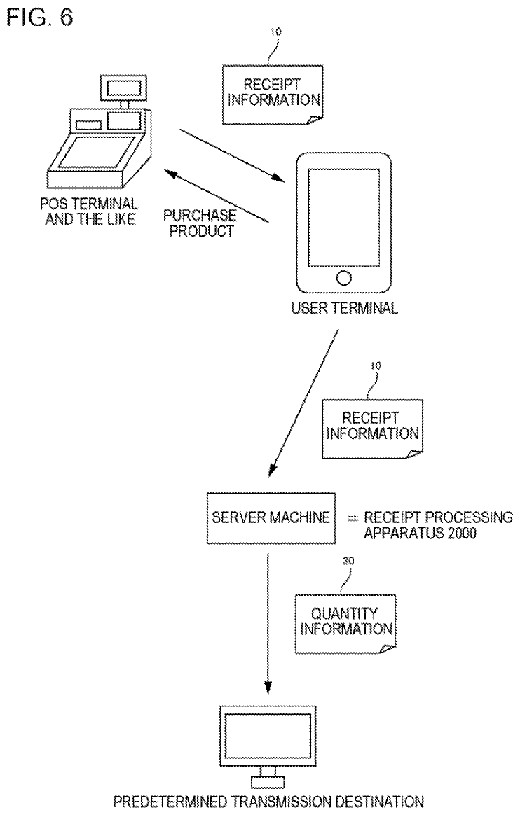

FIG. 6 is a second diagram illustrating a usage pattern of the receipt processing apparatus 2000. In this example, the receipt processing apparatus 2000 is a server machine. The server machine acquires the receipt information 10 for one or more users, and generates and transmits the quantity information 30 for each user. Specifically, a service that “quantity information 30 is generated from the receipt information 10, and the quantity information 30 is transmitted to a predetermined address” is achieved by the server machine on the Internet.

A user of the receipt processing apparatus 2000 registers use of the receipt processing apparatus 2000 in advance. Specifically, when a user purchases a product, the receipt information 10 indicating a result of the purchase is transmitted to the receipt processing apparatus 2000 in association with identification information of the user. Further, registration of the target product information 20 and a predetermined transmission destination is also performed. Thus, the target product information 20 and the predetermined transmission destination are stored in a storage apparatus in association with the user identification information.

Upon acquiring the receipt information 10 associated with the user identification information, the receipt processing apparatus 2000 generates the quantity information 30 from the receipt information 10. At this occasion, the target product information 20 registered in association with the acquired user identification information is used. Then, the receipt processing apparatus 2000 transmits the quantity information 30 to the predetermined transmission destination registered in association with the acquired user identification information.

In the case of the usage pattern illustrated in FIG. 6, the receipt information 10 may be transmitted from a user terminal to the receipt processing apparatus 2000, or may be transmitted from a POS terminal and the like for issuing the receipt information 10 to the receipt processing apparatus 2000. In the example of FIG. 6, the receipt information 10 is transmitted from a user terminal to the receipt processing apparatus 2000.

### <Acquisition of Receipt Information 10: S102>

The generation unit 2020 acquires the receipt information 10 (S102). As described above, the receipt information 10 is data indicating information relating to a purchased product. For example, the receipt information 10 is an electronic receipt, which is issued in place of a paper medium receipt or together with a paper medium receipt at the time of purchasing a product. Note that, a product herein also includes matter consumed at a purchasing place without being taken out, such as a meal eaten at a restaurant.

For example, the receipt information 10 indicates identification information (an identifier or a name), a purchasing price, a purchased quantity, and the like of a product, for each of the purchased products. The receipt information 10 may further indicate identification information of a user who purchased a product.

There are a variety of methods of acquiring the receipt information 10 by the generation unit 2020. For example, the generation unit 2020 acquires the receipt information 10 by accessing to any storage apparatus storing the receipt information 10. The storage apparatus may be installed inside the receipt processing apparatus 2000, or may be installed outside the receipt processing apparatus 2000.

In addition to the above, for example, the generation unit 2020 acquires the receipt information 10 by receiving the receipt information 10 transmitted from another apparatus. For example, in a case where the receipt processing apparatus 2000 is achieved by a user terminal, the receipt processing apparatus 2000 receives the receipt information 10 transmitted from a POS terminal and the like. Further, in a case where the receipt processing apparatus 2000 is achieved by a server machine, the receipt processing apparatus 2000 receives the receipt information 10 transmitted from a user terminal, a POS terminal, and the like.

In addition to the above, for example, the generation unit 2020 acquires the receipt information 10 by accessing to any storage apparatus storing the receipt information 10. For example, a case is proposed in which the receipt information 10 is managed in a database server, and a user who wishes to browse the receipt information 10 browses the receipt information 10 through a predetermined Web site or the like. In this case, the receipt processing apparatus 2000 may acquire the receipt information 10 by accessing to the database server.

There are a variety of timings at which the generation unit 2020 acquires the receipt information 10. For example, each time new receipt information 10 is generated, the generation unit 2020 acquires the receipt information 10. In addition to the above, for example, the generation unit 2020 may periodically (e.g., once a day) acquire the receipt information 10.

### <Acquisition of Target Product Information: S104>

The generation unit 2020 acquires the target product information 20 (S104). The target product information 20 is information indicating a target product included in the quantity information 30. For example, the target product information 20 indicates, as information representing a target product, identification information (such as an identifier or a name) of the target product. The information representing the target product may be information for determining one product, or may be information for determining a group of products (hereinafter, a product group). The information for determining a product group is, for example, identification information of a type of a product such as “tobacco”, “liquor”, or “carbonated drink”.

For example, the target product information 20 is stored in advance in a storage apparatus accessible from the generation unit 2020. The generation unit 2020 acquires target product information from the storage apparatus. Herein, in a case where the receipt processing apparatus 2000 generates the quantity information 30 for a plurality of users, target product information associated with user identification information is prepared for each user.

### <Generation of Quantity Information: S106>

The generation unit 2020 generates the quantity information 30 about a target product (S106). For example, the generation unit 2020 determines, from the product indicated in the receipt information 10, a product that coincides with a target product indicated in the target product information 20 by using the receipt information 10 and the target product information 20. Then, the generation unit 2020 generates the quantity information 30 about each of the determined one or more products.

### <<Determination of Target Product>>

As described above, in the target product information 20, information representing a target product may be information for determining one product, or may be information for determining a group of products (hereinafter, a product group). It is assumed that a target product represents one product in the target product information 20. In this case, the generation unit 2020 determines, from the product indicated in the receipt information 10, a product identical to the target product indicated in the target product information 20.

On the other hand, it is assumed that a target product represents a product group in the target product information 20. In this case, the generation unit 2020 determines, from the product indicated in the receipt information 10, a product belonging to the product group indicated in the target product information 20. For example, in a case where a target product indicated in the target product information 20 is “tobacco”, the generation unit 2020 determines, from the product indicated in the receipt information 10, one or more products belonging to the product group called “tobacco”.

Herein, information (hereinafter, group information) in which a product and a product group belonging to the product are associated with each other is used, for example, for determining whether a product indicated in the receipt information 10 belongs to a certain product group. For example, the group information is included in the receipt information 10. Specifically, each product is associated with a product group to which the product belongs in the receipt information 10. The generation unit 2020 determines a product belonging to the product group indicated in the target product information 20 from the product indicated in the receipt information 10 by comparing the product group indicated in the receipt information 10 with the product group indicated in the target product information.

The group information may be prepared separately of the receipt information 10. For example, the group information is determined in a database relating to a product. The generation unit 2020 determines a product group to which a product indicated in the receipt information 10 belongs by referring to the above-described database. For example, the generation unit 2020 is configured to be accessible to a product database managed by a POS system.

### <<Regarding Quantity>>

The quantity information 30 indicates a purchased quantity or a purchased frequency of a target product. In a case where the quantity information 30 includes a purchased quantity of a product, for example, the generation unit 2020 generates, as a purchased quantity of a target product, the quantity information 30 indicating the number of target products indicated in the receipt information 10. In addition to the above, for example, the generation unit 2020 may generate the quantity information 30 indicating a content quantity of a target product indicated in the receipt information 10. For example, in a case where a target product is a beverage, the quantity information 30 is caused to indicate how much milliliters the beverage has been purchased (e.g., in a case where two pieces of products each containing 500 ml are purchased, 1,000 ml). Further, the quantity information 30 may indicate, as a purchased quantity, both of the number of pieces purchased and a content quantity thereof.

In addition to the above, for example, the generation unit 2020 may compute, for each product that coincides with a target product included in the target product information 20 in the product included in the acquired receipt information 10, an integrated value of purchased quantities of the target product by further utilizing receipt information 10 other than the acquired receipt information 10. For example, the generation unit 2020 searches receipt information 10 that indicates the above-described identical products and that has been generated during a predetermined period, and integrates purchased quantities of the target product indicated in the acquired receipt information 10. Then, the generation unit 2020 generates the quantity information 30 in which the above-described integrated value computed for each target product is indicated as a purchased quantity of the target product. Note that, in this case, the quantity information 30 may indicate, for each target product, both of a purchased quantity of the product indicated in each piece of the receipt information 10, and an integrated value of purchased quantities of the product.

Also in a case where the quantity information 30 includes a purchased frequency, the generation unit 2020 determines, for each product that coincides with a target product included in the target product information 20 in the product included in the acquired receipt information 10, a purchased frequency of the target product by further utilizing receipt information 10 other than the acquired receipt information 10. For example, the generation unit 2020 searches receipt information 10 that indicates the above-described identical products and that has been generated during a predetermined period, and determines the number of pieces of the acquired receipt information 10. The number determined in this way represents a purchased frequency of the target product. The generation unit 2020 generates the quantity information 30 indicating a purchased frequency of each target product determined in this way.

The above-described predetermined period may be set as a fixed period common among all products, or may be set as a different period for each product. In the latter case, for example, the predetermined period is determined in association with identification information of a target product in the target product information 20. The generation unit 2020 utilizes the receipt information 10 generated during the predetermined period associated with a certain target product in the target product information 20 at the time of computing an integrated value of purchased quantities or a purchased frequency of the certain target product.

There are a variety of methods of determining the predetermined period. For example, the predetermined period is determined by a start point and an end point of the predetermined period (“from 11/1/2018 to 11/30/2018” and the like). In addition to the above, for example, the predetermined period is determined by a start point and a duration of the predetermined period (“30 days from 11/1/2018” and the like). In addition to the above, for example, the predetermined period may be determined as a period having a predetermined duration in which a time when processing by the generation unit 2020 is performed is set as an end point such as “one month in the past”. Further, the predetermined period may be determined as a repetitive period such as “from a first day to a last day of every month”.

The above-described search of the receipt information 10 is performed, for example, with respect to a database for managing the receipt information 10. However, the receipt information 10 does not have to be necessarily managed by the database. For example, search of receipt information 10 that satisfies a certain condition may be achieved by acquiring all pieces of receipt information 10 from a storage apparatus storing the receipt information 10, and determining whether each piece of the receipt information 10 satisfies the condition.

Herein, for computing a purchased quantity and a purchased frequency, in addition to setting, as a search target, the receipt information 10 representing purchase by a user of the receipt processing apparatus 2000, receipt information 10 representing purchase by another person associated with the user may be further set as a search target. This is because a person who purchases a product used by a user of the receipt processing apparatus 2000 is not limited to the user. For example, a housemate of a user is associated with the user of the receipt processing apparatus 2000. Information representing a correlation between a user and the other person as described above is stored in a storage apparatus accessible from the receipt processing apparatus 2000.

<<Regarding Case where Condition is Set for Generating Quantity Information 30>>

The generation unit 2020 may include, in the quantity information 30, a quantity relating to a target product, only in a case where the quantity relating to the target product satisfies a certain condition. For example, the condition is a condition that “a quantity relating to a target product is equal to or more than a threshold value” and the like.

For example, the above-described predetermined condition is included in the above-described target product information in association with identification information of a target product. The generation unit 2020 determines whether the predetermined condition associated with the target product is satisfied, when the target product is included in the receipt information 10. When the predetermined condition is satisfied, a quantity relating to the target product is included in the quantity information 30. On the other hand, when the predetermined condition is not satisfied, a quantity relating to the target product is not included in the quantity information 30.

The above-described predetermined condition may be a condition on a quantity during the above-described predetermined period. For example, the predetermined condition is a condition that “a purchased quantity during one month in the past is X or more”. In this case, when a purchased quantity or a purchased frequency of a target product computed by using the receipt information 10 generated during the predetermined period satisfies the predetermined condition, the generation unit 2020 includes the quantity in the quantity information 30.

Including, in the quantity information 30, only a quantity relating to a target product that satisfies a condition as described above enables to appropriately narrow down information to be transmitted. For example, in a case of liquor, too much drinking may be harmful to health, but drinking modestly may not cause health problems. In view of the above, in a case where a purchased quantity and the like of a certain product is more than an allowed quantity, the quantity information 30 relating to the product is transmitted.

### <Transmission of Quantity Information 30: S106>

The transmission unit 2040 transmits, to a predetermined transmission destination, the quantity information 30 generated by the generation unit 2020 (S106). The transmission destination of the quantity information 30 may be one or more. Information indicating the predetermined transmission destination is stored in advance in a storage apparatus accessible from the transmission unit 2040. The transmission destination of the quantity information 30 can be represented by any data usable for representing a transmission destination of data, such as a mail address and an IP address.

The transmission destination of the quantity information 30 may differ for each product. For example, a transmission destination of the quantity information 30 relating to a product is associated with each product in target product information. In this case, the generation unit 2020 generates the quantity information 30 for each product associated with a same transmission destination. Then, the transmission unit 2040 transmits each piece of the quantity information to the transmission destination of the quantity information 30.

### Example Embodiment 2

### <Overview>

In an example embodiment 2, quantity information 30 indicates, as a quantity relating to a target product, a predicted consumption quantity of the product by a user. In a case where a user lives together with another person (e.g., a family member), it is conceived that, depending on a type of a product, a product is consumed not only by the user alone, but also by a plurality of persons. Therefore, it is conceived that a quantity of a certain product consumed by a user is less than a purchased quantity. In view of an aspect of health management and the like of a user, it is conceived that recognizing how much the product is consumed by a user is more important than the purchased quantity of the product.

In view of the above, a receipt processing apparatus 2000 computes a predicted consumption quantity of a target product by a user, based on a purchased quantity of the target product indicated in receipt information 10. Herein, the receipt processing apparatus 2000 acquires conversion information for use in computing a predicted consumption quantity from a purchased quantity. For example, the conversion information indicates a ratio of a predicted consumption quantity to a purchased quantity. The receipt processing apparatus 2000 computes a predicted consumption quantity of a target product by using a purchased quantity of the target product indicated in the receipt information 10 and the conversion information. For example, it is assumed that the conversion information indicates one-third, as a ratio of a predicted consumption quantity to a purchased quantity. In this case, a generation unit 2020 computes a predicted consumption quantity by multiplying one-third by the purchased quantity of the target product indicated in the receipt information 10. Then, the receipt processing apparatus 2000 generates quantity information 30 indicating, as a quantity relating to the target product, the predicted consumption quantity of the target product.

### Advantageous Effect

In the receipt processing apparatus 2000 according to the present example embodiment, a predicted consumption quantity of a target product by a user is computed from a purchased quantity of the target product indicated in the receipt information 10, and the predicted consumption quantity is transmitted to a predetermined transmission destination. Thus, the predicted consumption quantity of the target product by a user can be easily managed. For example, transmitting, to a hospital and the like, a predicted consumption quantity about a target product associated with health management and promotion of a user enables to more easily and accurately perform health management/promotion of the user.

In the following, the receipt processing apparatus 2000 according to the present example embodiment is described in more detail.

### Example of Functional Configuration

Similarly to the functional configuration according to the example embodiment 1, a functional configuration of the receipt processing apparatus 2000 according to the example embodiment 2 is illustrated in FIG. 2, for example.

### Example of Hardware Configuration

Similarly to the hardware configuration of the receipt processing apparatus 2000 according to the example embodiment 1, a hardware configuration of the receipt processing apparatus 2000 according to the example embodiment 2 is illustrated in FIG. 3, for example. However, a storage device 1080 constituting the receipt processing apparatus 2000 according to the example embodiment 2 stores each program module for achieving a function of the receipt processing apparatus 2000 according to the example embodiment 2.

### <Flow of Processing>

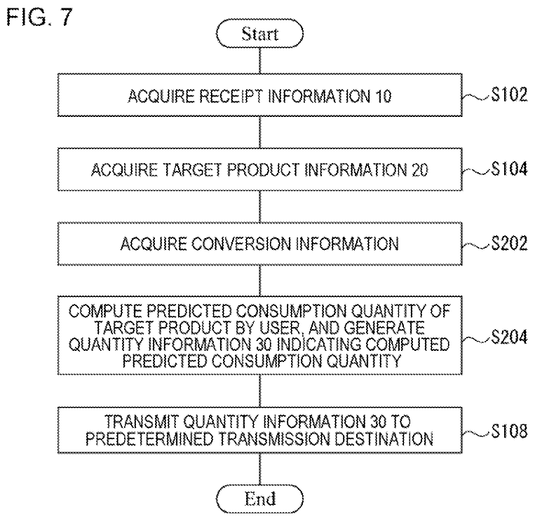

FIG. 7 is a flowchart illustrating a flow of processing to be performed by the receipt processing apparatus 2000 according to the example embodiment 2. Note that, S102, S104, and S108 are similar to those in the flowchart of FIG. 4. The generation unit 2020 acquires conversion information (S202). The generation unit 2020 generates the quantity information 30 by computing a predicted consumption quantity of a target product included in the receipt information 10 (S204).

### <Acquisition of Conversion Information: S202>

The generation unit 2020 acquires the conversion information (S202). As described above, the conversion information is information for use in computing a predicted consumption quantity from a purchased quantity. The conversion information may be determined for each target product, or may be common among all target products. In the former case, the conversion information may be included in target product information 20. Specifically, the conversion information indicates information for converting a purchased quantity of a target product into a predicted consumption quantity in association with the target product in the target product information 20. In this case, S202 is included in S104 in FIG. 7.

In a case where conversion information is acquired separately of the target product information 20, there are a variety of methods of acquiring the conversion information by the generation unit 2020. For example, the generation unit 2020 acquires the conversion information by reading the conversion information from a storage apparatus storing the conversion information. In addition to the above, for example, the generation unit 2020 may acquire the conversion information by receiving the conversion information transmitted from another apparatus.

### <Computation of Predicted Consumption Quantity Utilizing Conversion Information: S204>

The generation unit 2020 computes a predicted consumption quantity of a target product by a user by utilizing the conversion information (S204). The conversion information indicates various pieces of information for use in converting a purchased quantity of a target product into a predicted consumption quantity. For example, as described above, the conversion information indicates a ratio of a predicted consumption quantity to a purchased quantity about the target product. In this case, the generation unit 2020 computes a predicted consumption quantity of the target product by a user by multiplying the ratio indicated in the conversion information by the purchased quantity of the target product.

In addition to the above, for example, the conversion information may indicate a total number of persons including a user of the receipt processing apparatus 2000 and a housemate of the user (such as a family member). In this case, the generation unit 2020 computes a predicted consumption quantity of the target product by a user by dividing a purchased quantity of the target product by the above-described total number of persons.

In addition to the above, for example, the conversion information may indicate an attribute of each of a user of the receipt processing apparatus 2000 and a housemate of the user. For example, the attribute indicates sex, age, a body shape, a preference, and the like. Information relating to the body shape indicates, for example, information such as height, weight, and the like. Information relating to the preference indicates information such as likes and dislikes of groceries, smoker or non-smoker, and the like.

For example, the generation unit 2020 computes an index value representing a magnitude of a predicted consumption quantity of a target product, based on an attribute indicated in the conversion information for each of a user and a housemate of the user. In the following, the index value is referred to as a consumption index value. For example, the consumption index value is computed by the following equation (1).

$$\begin{matrix}[ Formula 1 ] & \\ P ( i , j ) = {\sum }_{k} a ( j , k ) * p ( i , k ) & ( 1 )\end{matrix}$$

Herein, i represents an identifier of a product, j represents an identifier of a person, and k represents an identifier of an attribute. P(i,j) represents a consumption index value relating to a product i regarding a person j. a(j,k) represents a value of an attribute k regarding the person j. p(i,k) represents a magnitude of influence of the value of the attribute k on a consumption quantity of the product i.

The generation unit 2020 computes, as a ratio of a predicted consumption quantity of a user to a purchased quantity of a target product, a ratio of a consumption index value of the user to a sum (=ΣP(ij)) of the computed consumption index value P(i,j). Then, the generation unit 2020 computes the predicted consumption quantity by multiplying the ratio by the purchased quantity of the target product.

The conversion formula such as the equation (1) may be included or may not be included in the conversion information. Likewise, the magnitude p(i,k) of influence of the value of the attribute k on the consumption quantity of the product i may be included or may not be included in the conversion information. The conversion formula and p(i,k) that are not included in the conversion information are, for example, set in advance in the generation unit 2020. In addition to the above, for example, these pieces of data may be stored in a storage apparatus accessible from the generation unit 2020 separately of the conversion information.

While the example embodiments of the present invention have been described with reference to the drawings, the example embodiments are only exemplification of the present invention, and combination of the above-described example embodiments, or various configurations other than the above can also be employed.

A part or all of the above-described example embodiments may also be described as the following supplementary notes, but the present invention is not limited to the following.

1. A receipt processing apparatus including:

a generation unit acquiring target product information indicating a target product, and receipt information relating to a purchased product, and generating, from a product indicated in the receipt information, quantity information indicating a purchased quantity or a purchased frequency of a target product indicated in the target product information; and

a transmission unit transmitting the quantity information to a predetermined transmission destination.

2. The receipt processing apparatus according to supplementary note 1, wherein

the generation unit computes an integrated value of a purchased quantity of the target product indicated in each of a plurality of pieces of receipt information generated during a predetermined period, and uses the integrated value, as a purchased quantity of the product indicated in the quantity information.

3. The receipt processing apparatus according to supplementary note 1 or 2, wherein

the transmission unit transmits the quantity information, in a case where a purchased quantity or a purchased frequency of the product satisfies a predetermined condition.

4. The receipt processing apparatus according to supplementary note 3, wherein

the predetermined condition is determined for each product.

5. The receipt processing apparatus according to any one of supplementary notes 1 to 4, wherein

the generation unit computes, from a purchased quantity of the target product indicated in the receipt information, a predicted consumption quantity of the target product by a user by using conversion information for computing a predicted consumption quantity from a purchased quantity of a product, and generates the quantity information indicating the computed predicted consumption quantity.

6. The receipt processing apparatus according to supplementary note 5, wherein

the conversion information indicates a ratio of a predicted consumption quantity of the user to a purchased quantity about the target product, and

the generation unit computes a predicted consumption quantity of the target product by a user by multiplying the ratio indicated in the conversion information with respect to a purchased quantity of the target product.

7. The receipt processing apparatus according to supplementary note 5, wherein

the conversion information indicates a total number of persons including the user and a housemate of the user, and

the generation unit computes a predicted consumption quantity of the target product by a user by dividing a purchased quantity of the target product indicated in the receipt information by the total number of persons.

8. The receipt processing apparatus according to supplementary note 5, wherein

the conversion information indicates information relating to an attribute of the user and a housemate of the user, and

the generation unit computes a predicted consumption quantity of the target product by a user by using the attribute indicated in the conversion information.

9. A control method executed by a computer, the method including:

a generation step of acquiring target product information indicating a target product, and receipt information relating to a purchased product, and generating, from a product indicated in the receipt information, quantity information indicating a purchased quantity or a purchased frequency of a target product indicated in the target product information; and

a transmission step of transmitting the quantity information to a predetermined transmission destination.

10. The control method according to supplementary note 9, further including

in the generation step, computing an integrated value of a purchased quantity of the target product indicated in each of a plurality of pieces of receipt information generated during a predetermined period, and using the integrated value, as a purchased quantity of the product indicated in the quantity information.

11. The control method according to supplementary note 9 or 10, further including

in the transmission step, transmitting the quantity information, in a case where a purchased quantity or a purchased frequency of the product satisfies a predetermined condition.

12. The control method according to supplementary note 11, wherein

the predetermined condition is determined for each product.

13. The control method according to any one of supplementary notes 9 to 12, further including

in the generation step, computing, from a purchased quantity of the target product indicated in the receipt information, a predicted consumption quantity of the target product by a user by using conversion information for computing a predicted consumption quantity from a purchased quantity of a product, and generating the quantity information indicating the computed predicted consumption quantity.

14. The control method according to supplementary note 13, further including:

providing the conversion information that indicates a ratio of a predicted consumption quantity of the user to a purchased quantity about the target product; and

in the generation step, computing a predicted consumption quantity of the target product by a user by multiplying the ratio indicated in the conversion information with respect to a purchased quantity of the target product.

15. The control method according to supplementary note 13, further including:

providing the conversion information that indicates a total number of persons including the user and a housemate of the user; and

in the generation step, computing a predicted consumption quantity of the target product by a user by dividing a purchased quantity of the target product indicated in the receipt information by the total number of persons.

16. The control method according to supplementary note 13, further including:

providing the conversion information that indicates information relating to an attribute of the user and a housemate of the user; and

in the generation step, computing a predicted consumption quantity of the target product by a user by using the attribute indicated in the conversion information.

17. A program causing a computer to execute each step of the control method according to any one of supplementary notes 9 to 16.

This application is based upon and claims the benefit of priority from Japanese patent application No. 2018-238152, filed on Dec. 20, 2018, the disclosure of which is incorporated herein in its entirety by reference.

### REFERENCE SIGNS LIST

10 Receipt information
20 Target product information
30 Quantity information
1000 Computer
1020 Bus
1040 Processor
1060 Memory
1080 Storage device
1100 Input/output interface
1120 Network interface
2000 Receipt processing apparatus
2020 Generation unit
2040 Transmission unit

What is claimed is:

1. A receipt processing apparatus comprising:
a generation unit that acquires target product information indicating a target product, and receipt information relating to a purchased product, and generates quantity information indicating a purchased quantity or a purchased frequency of the target product indicated in the target product information out of the product indicated in the receipt information; and
a transmission unit that transmits the quantity information to a predetermined transmission destination.

2. The receipt processing apparatus according to claim 1, wherein
the generation unit computes an integrated value of the purchased quantity of the target product indicated in each of a plurality of pieces of receipt information generated during a predetermined period, and uses the integrated value, as the purchased quantity of the product indicated in the quantity information.

3. The receipt processing apparatus according to claim 1, wherein
the transmission unit transmits the quantity information, in a case where the purchased quantity or the purchased frequency of the product satisfies a predetermined condition.

4. The receipt processing apparatus according to claim 3, wherein
the predetermined condition is determined for each product.

5. The receipt processing apparatus according to claim 1, wherein
the generation unit computes, from a purchased quantity of the target product indicated in the receipt information, a predicted consumption quantity of the target product by a user by using conversion information for computing a predicted consumption quantity from a purchased quantity of a product, and generates the quantity information indicating the computed predicted consumption quantity.

6. The receipt processing apparatus according to claim 5, wherein
the conversion information indicates a ratio of a predicted consumption quantity of the user to a purchased quantity about the target product, and
the generation unit computes the predicted consumption quantity of the target product by a user by multiplying the ratio indicated in the conversion information with respect to the purchased quantity of the target product.

7. The receipt processing apparatus according to claim 5, wherein
the conversion information indicates a total number of persons including the user and a housemate of the user, and
the generation unit computes the predicted consumption quantity of the target product by the user by dividing the purchased quantity of the target product indicated in the receipt information by the total number of persons.

8. The receipt processing apparatus according to claim 5, wherein
the conversion information indicates information relating to an attribute of the user and a housemate of the user, and
the generation unit computes the predicted consumption quantity of the target product by the user by using the attribute indicated in the conversion information.

9. A control method executed by a computer, the method comprising:
acquiring target product information indicating a target product, and receipt information relating to a purchased product, and generating quantity information indicating a purchased quantity or a purchased frequency of the target product indicated in the target product information out of the product indicated in the receipt information; and
transmitting the quantity information to a predetermined transmission destination.

10. The control method according to claim 9, further comprising
in the generating, computing an integrated value of a purchased quantity of the target product indicated in each of a plurality of pieces of receipt information generated during a predetermined period, and using the integrated value, as the purchased quantity of the product indicated in the quantity information.

11. The control method according to claim 9, further comprising
in the transmitting, transmitting the quantity information, in a case where the purchased quantity or the purchased frequency of the product satisfies a predetermined condition.

12. The control method according to claim 11, wherein
the predetermined condition is determined for each product.

13. The control method according to claim 9, further comprising
in the generating, computing, from a purchased quantity of the target product indicated in the receipt information, a predicted consumption quantity of the target product by a user by using conversion information for computing a predicted consumption quantity from a purchased quantity of a product, and generating the quantity information indicating the computed predicted consumption quantity.

14. The control method according to claim 13, wherein
the conversion information indicates a ratio of a predicted consumption quantity of the user to a purchased quantity about the target product, and
the method comprises, in the generating, computing the predicted consumption quantity of the target product by the user by multiplying the ratio indicated in the conversion information with respect to the purchased quantity of the target product.

15-16. (canceled)

17. A non-transitory computer readable storage medium storing program causing a computer to execute the control method comprising:
acquiring target product information indicating a target product, and receipt information relating to a purchased product, and generating quantity information indicating a purchased quantity or a purchased frequency of the target product indicated in the target product information out of the product indicated in the receipt information; and
transmitting the quantity information to a predetermined transmission destination.
